SEQUENCE LISTING (4 sequences)

SEQ ID NO: 1 - DNA, 2854 bp, Bacteroides fragilis
  ref <301>: AUTHORS: Muphy, C. G. and Malamy, M. H.
  ref <302>: TITLE: Characterization of a ′mobilization cassette′ in transposon Tn4399 from Bacteroides fragilis
  ref <303>: JOURNAL: Journal of Bacteriology
  ref <304>: VOLUME: 175
  ref <305>: ISSUE: 18
  ref <306>: PAGES: 5814-5823
  ref <307>: DATE: 1993
  ref <308>: DATABASE ACCESSION NUMBER: L20975
  ref <309>: DATABASE ENTRY DATE: 2003-11-25
  ref <308>: DATABASE ACCESSION NUMBER: L20975
  ref <309>: DATABASE ENTRY DATE: 2003-11-25
  ref <313>: RELEVANT RESIDUES IN SEQ ID NO: (1)..(2854)
tatgctacccattcatcaaatcatcgttttttcactcatcactaaatccgtagtaaattatacaatgaacatacaggaagcaaaagcaatcagattggcagactatctgcaaagtttgggctattcacccgtcaagcaacagggaaacagcctttggtacaaatctcctctcagggaagaatccgaaccgtcattcaaggtcaatgctgaattgaacagatggttcgattttggactcggcaaaggcggcaatatactcgcactggcaaaagaactctatcaggacgattatgtaccctcgctgcttgccaaaatagcagggcaggcaccgcatattcgccccacatcgttttcttttcgtcagcaaccgtccgaaccgagttttcaacagttggaagtcagggagcttgcgcacccctgcactgctccgatacttgcaggaacgaagtattgatacggatattgccaagagcgaatgtaaagaacttcacttcacccataacggcaaaccttactttgccatcggctttgagaacgtgacaggtggttatgaggtgcgcaatccgttctttaaaggctgtgtcgctccgaaagacatcacgcacattcggcaacaggaacgacaggcagatacttgctatctgttcgagggcttcatggactatctttcattcctgactctccgtaagcagaagcaaccgcaatattccggcttgcaaggacaggactacatggttctcaactctgtttccaatcttggcaaggctatggacaggttgtcggactatgagcgcatccactgttttttcgacaacgaccaagcaggaaacaaggcttgtttggaattgcaacgcacgttctcctttcgggttagggatgcttccatccactattccgagtacaaggatttgaacaatttcctatgtggcaggaaagcggtggaggataagaaaaatgaggttttagttcgaccaaaacccaagaggaagggattcggcatctaagccgtaaatcacatcttttttaggatagcaagttaatgtcggcgtattacattccccgaccacttgcccctcaaaaagatgaggggaacggctcccgatggtcacggacttttcagattacatcaatcggattattcactaaaaaacagatagaacagatgaagaaaaacaacataatcaccttacgtctgacagacgatgaactcggttggctgggtgcattgtgcagacggagcagacagagtaaaagcgaaaccatccgttcgctcatcatgaacggcacggtacgggaacgaatcaataaagaacaccttgccgtcatccggcagttgattggcgagagtaccaatctcaaccagttggcaagacaggcgaacacctacggattctttgccgtagccgataagtgcgagaacatggcaaaggcagtgtcccaactcataaacaggctgaaagatgataggtaagcagacgaaaggaacatctttcagcggttgcgtatgctatgtgctgagagaggacaaatccaagttgctggaagctgttggcgtggagggaacgcccgaacagatggcggagcagtttgagttgcagacattgctcaatgacaaggtaaagaacacggtcggacatacctcgctcaacttctcacccgaagacggtgagagattaaagacagacgatgcactcatgttgcagatagcccatgattacatggcgaagatggggatagaaaatacgcagtacattgtagcccgacataccgacagggagcatccgcactgccacatcgttttcaaccgtgtggacaatgacggtaagactatcagcgacaagaacgatttttaccgcaacgagaaagtctgcaagatgcttaccgccaaatacaggctgcatttcgccaacggtaaggataacatcaaggaagaacgcttgcgtccatacgacaaggctaagcatgaggtttacaaggcattgaaaggggaactgccccacgcccgaagttggaacgaactgaaagaagctctttccgaacgggacattgaattgaagttcaaggtgagccggacaacaaaagaggtgcagggcgtgaagttcgagtatggcggcatatccttttccggctcaaaggtcagtcgggagttcagttacatgaacatcgactaccggttaaggcggaatgcctttgaggatgatttcaatcatagacaggcagctatccgggaaccgggagaggaagcagtacagactgtatcgcaaaaccgttccaaagatagcgtcggtaatggattgggattattcagcggtataggctcttcttttgatgtggcagatgcagaagccaaccaagaaatggcggagatactacgaaagaagaagaaagccaagcgcaagcgtggcatgagattataacgacttatttatcaacttaaaagattaactacaatggtacagaaagactttttattggattttgacctctacatttgtgagaggttcggttacagaaacagcctgcacgtgacacagacaatgaacggtttttgcgtggatgtgaacatcaagcccgtaaaactctacatccgtttttgggagtacagcaatggcgtgggcggttttcccgactggtgcatcatccttgtgcgttgcaactttgccaaaaaacaggaacaggaattgcaggaacttgtgcggttcttcaaggagtatgcgccacaatatgggtacaagtatatcgggattgaggataacactcaactaaatcaaatattagagtttcagcgcatcctaatgagaggacaggatagctttgtttttttcattgacacaatatgagcggcataaaagct

SEQ ID NO: 2 - DNA, 12105 bp, Bacteroides fragilis
  ref <301>: AUTHORS: Tribble, G.D., Parker, A.C. and Smith, C.J.
  ref <302>: TITLE: The Bacteroides mobilizable transposon Tn4555 integrates by a site-specific recombination mechanism similar to that of the gram-positive bacterial element Tn916
  ref <303>: JOURNAL: Journal of Bacteriology
  ref <304>: VOLUME: 179
  ref <305>: ISSUE: 8
  ref <306>: PAGES: 2731-2739
  ref <307>: DATE: 1997
  ref <308>: DATABASE ACCESSION NUMBER: U75371
  ref <309>: DATABASE ENTRY DATE: 2005-06-06
  ref <308>: DATABASE ACCESSION NUMBER: U75371
  ref <309>: DATABASE ENTRY DATE: 2005-06-06
  ref <313>: RELEVANT RESIDUES IN SEQ ID NO: (1)..(12105)
tgtatttataaccatagaacaaccaaaactaatcagaaatatccgagtgtaaatctttaattaccagcatattctaaaatttaacacttttcggttgccatttgtcgcttcccaaatttccacatatttttgagacgtatttctgacgtggagaatttgcggggcttgttttttgtcacaccgtgcagacagttgacaagggtttacaaccgtttacgacaagcgacaataaccgccccgatatgcggaaacagtcacactgtgtagaaaagcgacaatggttgacttccgttcacatccgtttaccatttcagagatataggattgaagaaatgaaccattaaacgataaaacggtatgaaagcaaccagaaaatgcagtttttgcggcaagtcctttgtaacccgaagcggtatgcaaagatattgcagtgaggcttgtcaggcagaagccaaacgagccagagtgatgcagaagaacaacctcttcaaagtcgcccaacccttgatggagatacagcatcaggagtatctcaccttttccaaagcagccatcctcatgggctgttcccgacagtacatttacaaacttgtagccatcggcaagctgaaagcctcacgcatcagcaaccgcatggcattcatccgcagagccgacatcgagcagatgttggagggcaatccctatcaccgcatcctgcccggcaacacctccacaccaaggaaatcatcttcatcttccttacctgccaaaagagaaaaaagggaaaaggaaagcgaagaagtgttggacttctattcgggcgaggaggtgatgtccctttttaaggtaaagcagtcatggctttacacttccgccaagcgtaaccatatccccatctgccgtatcgcaggaaagaactattacagcaagaagcatattgacgagtttttcggtgtggcagttgatattagcgaaattaccgactggctactgaccgaggaggtggaggaactgttcggcatgaagccgaccgcactccgtgcctacacctatcgccataagatacccactaaaagagagtacgggcgtacctattactccaaatcacatttgaacgaactccgcagaactgaccttgtgaacgatgaacgctactataccgttgagcaggtgcagcaaatctatggtctttcgtcagccaacatctgccatatcgtcaaggtgaagcacatcgaaaagataaaggtgggtgtgaaaaacctgcttttgcgctcagatgtggagcgtgtcatggctgaaaggaacaaataaccgtgaaaaatcgggattatcgaaaattatttcaaaatgatgtatcgtggaggtattcacggatatttcacgttgttcctttgccatcggaaacatggatacaactccaaaatgtatacaaccaattaaaacataattattatgagtaaatgcaaaacagttaccttgcgtaagcgcaagattaagaacgggacacagtattcactatgccttgactactatcccggctaccgtgacaatgtcaccatgagagtgattacacgtgaagccttaggaatttacatcttcgccaaacctgcaaaccagcaggaacgggacttcaacgcacgcatgatgaagaaagcggtcatcctgcgcaaccagcgctacgaagccattttcaatgaaaacaacggcttttttgacaagaccaagatgaagggcgatttccttgcctatttcaaaggactggctgaccgcaagaatatcaagtggcagcacgtatacaagcatttccagcggttcgtgaacggcaaatgcacctttgaggaggtggatgtggatttgtgccgcaagttcatggaatacctgcttgatgcaccccaatccatccacaccaaccaaaagctgcacatcaactccgcagcaggctattggtcaactttccgtgccgtgctgcacaccgcctaccgtgacaggaagataaaggagaacccaaacggcttcttagaccgcatcgagtgcattcccaccatcagggagcatttgagccaagaggaactgatacggcttgccgaaacaccctgtgaggaggaggtcttgaaaaaagcttttcttttcgcctgtcttacgggactgagaaagagcgacatcagacagctcacgtggcagcagatacaaccatacaccaacggcaggatgttcgttaccacccgtatgcagaaaaccaaagaaatagtgcataaccccatcagtgatgaagcctatggactgctgggagaacggggcgagggacttatctttgaggatttcaaggacaagatgctgcaaggaccactccaacggtggctcacggcagcagggataaccaagaaaatcacctttcactgtacccgccacagcttcggaagcctgcacgtggaaatgggaacggacatggctgtcatccaagcctatctcggacataagaacattaccaccacacaaatctattccaagatagcagcgcagcagatgtgtcaggtggtggacaagataaccttgaagcgcaaggaggcataaatgtctcacattgacaggtattcagaggggcggttatggctacaaggctataatcgcccctctatgtttttggcttgatgccaccatttataagcccctcagagtatgaaacagagtatgatatgatgacatttcgtgaaatacattgaaaaagaccgttctaaccgcaaataacgggcagtaattgggaaaaatagcattaactttgcacaaaataatacaggaacattcaatctctcaacgaaatatggaatcatcaatcaaggacaaatacatcatcttgggctttgtcggcttcgccatcgtcctaatatcttccattgccacgctggtaatagcggacagcttcaaccaagacaactttgtcaggtggatagtattcgtatgctgtaacctgttgggatggttgctctatctctcctttcagacacttatctttgatacatacgaaatctacaaaatcaagttcggcaagaaagaaacgattgccgaagccatagaggtgcaggaagaactgtcacaaaatacacttgaagaagccacatctgtgcctggacctacatcagtccctgagcctgtacccgaatcatccccgacaaaagaagagacacttatccaaacacaaccgatagagcttactatcgccccggatcttcacgaaaagaaccgtgccaattacgcaagcagagagcaacgggaaaaggaagagcgcatccgcatggtcatggagtattgccattattacctgcctcgcattgccgaccaagaaaccgtgaaccacatctgtactgaggtggacaaatggatgaatcttaacacttataccccgaagcccatacaaagaccgtttaccaaagacatcaacaacattccactccgtcacttcgtatggaatatctctgagcgtttcctgtacaagagatactacaatggggataaccgtgccaagttcatcaaagcccttttcccgaaatcgtttgctgatacagacttatcaaccatcaagaatttcaaggtagagccgttaaagacggaaattcccattgatgaacccgaaaacggcaaacttgatttccactatcccgaggattatgtgcggaattaggataatcacgacaccaacgacaaccatccggtaactcataaccgcctgttttacatcgtttcccgagtaattttacccgtcatttatggctgggcttaattattcgggaattattttgcaatgacgtgtcgtggagatattcacggatatatcatttcagtcctttgcgccaagtcttacaaaagagacgagtacgcgaatggaaaaatcaattcttaccttcaacgacctccccgaggttgtcgctcagcttcgagacgaagtgatgagcctgaaaagcctgctcgccgagcagcgcagtgtgaacaatgccaaaacggtggacacccacgtgcccatgtctgtggacgaggcagcagagtatttaggtatccctaagggtacgctctacatgaaactgtcagaagggacaatccctgccaccaagcccggcaaacgctattgcctttaccgtgacgaactggacaagtggctggaaaccgcccgaaagaatcccataccgttgtcagacgaggaactgaacaagtccttatcctcttcccaccgtcgcaagcccaacccacgtaactggtgaatgatatggaagaggataagaactatatcaacctgatacgtggcgacctcacaaaagcatcccaagcgcataacggtatgcccgacagtgtaggcatgatgaatatcaagacggcaaaccaaaccattcttgaagcatcgttattgcctacgccccgtgcgctgtgggacagcttttggtacgagggggaactctcctgcttgtttgccgattccaacgtgggcaagtccatccttgccgtgcagatagccgaccgcatcgcccgaaccgacaatgtgctgtatctggactttgaactgtccgaaaagcagttccagctccgctataccaacgagcatggagagctctacacctttcccgacaaaatctatcgggtgtctattgactgcaaccagcttttggatgccaactttgaggaagctatcataggcggcattgaacagatggctgtgcagaccgactgcaagattttcatcattgacaatcttacctacctgtgttgcgccatggagaaaggcgatgccgcaggacggctgatgattcagctgaacaatctcaaaaagagatatgcgctctctatccttgtcctggcacatacgcccaaacgctctttggattgtcccatcacatccaacgaccttgccggaagcaaacggctctacaatttctttgacagcgtgttcaccattggaaaaagtgcccaagacggagggcttcgctatgtgaagcagcttaaagtgcgctatggcacgttctctcatgatgcggataatgtaatcgtttacgagattgacaaggtggatgctttcttgcagttcgtgttcaggggctattccacggaaaaggaacacttgaaaaaattgggcgacaatgaatcaagccaaagggattgccaaattctgcaactctcccaatcgggcaagtccgtcagggagatagcctcacaggtgaattgtggcaagtccaccgtaaaccgtatcatccagcgcagcaaagagagtaaaaacgcaggtgtcccaagtgtcccactgtcccaacccttagagtgtgggacaatgggacaggatgggacagccgacaatcaaccatcaaaaacggactaagctatgggcaattattcattacagaagtataaaggaacggcaacacggcatacctgccccaaatgcggagacaggcattctttcgtctattacgtggacgaaaataatgtgccgttgcatccatcggtcggcagatgtaaccacgaaagcggttgtgggtatcactacactccgaaagagtattttcaagagcatcctgaacacagaactaccaatgatttctcttttgacaggcaaagagcagagcagaagaaagtgaagcagcaaagtaagccgacagccatcggctatattccccctcactatgtggagaagtcgcaaagcgagcgtagcaatttcttccgtttcctcttcacactccttacttcctactatggcgacaaggcgaaagaggtgttgaagcggttgttggaggaataccgtttgggggctacccgtgacggctctgttatcttttggcagatagacaggacgggcaaggtacgcacgggaaaggtgatgcagtacaatcccgaagacggacaccgtatcaagggaggacagacatcggcagtgaactggatacacagcatattgaaaaagcagcgtgtgttggcagaggattggcaactatcccaatgccttttcggggaacacttgttgaaaacgcatcccgacaaggtggtggtcttggtggaatccgagaagagtgccgttatcggttctgctatcttccccgattatgtatggctggctacgggtggtaagagtcagatgagagaagagaaactccgtgtactgtcagggcgaaccgtgcttctctttcccgatgccgatgcttatgccgagtggaaacagcgagccgagagcatgtacttttgtaaggtggtggtttcggacatcatcgaaaggaatgccaccccgaaacaaaaagaagcccatatcgacatagccgattggattatctttcagatacgggagggcaaggtgatgagtacagccaaccacttggtcgaggctgagagaatcctccagcggatgatagagaagaatcccgtcctgcaaaaactgatagacgatttagaccttgtgctggtcggtgcatctccaatcggcaacgatgatgaaaaacctccctgacggaggagagcggaagccgtaggctggagtttgcagacaatggcttgccattgatatagcccactataactacacgctccgcttacgtagttgtgggctctcccgaggggattagggttttaccctaatgacccactcagggcgtttctcccctgagaacccagagcaaagagtgaccctctctttgcaatctccgcttatgggttgcacccctaagaaccccatgcgtttacggacagcggaaaagcaaacaataaagtacaaacccaaaaaacaagtatctatggcaacaaaatcaagcatacatatcaagccctgcaatatcgcatcgagtgaggctcacaacaggaggactgccgaatacatgcgccacatcggagagtccagaacctatgtggttccagaactctccaccgataatgagcagtggataaatcccgacttcggcagtccggatttgcggatgcattatgacaatatcagacagatggtaaaggaaaagaccggacgtgccatgcaggaaaaggagcgtgaacgcaaaggcaagaacggtaaaatagtcaagattgcgggatgctcccccatacgtgaaggagtgctgcttgtcaggtcggacaccacactggcagacgtgcgtaaattcggtgaggagtgtcaaagacgctggggaatcacaccgctgcaaatcttcctgcacaaggatgaagggcattggctgaacggtcagccggaagcggaagacagggaaagcttcaaagtcggggacagatggttcaagccgaactatcatgcccatatcgttttcgactggatgaaccacgaaacaggaaagagccgaaagctcaatgacgatgacatgatgcagatgcagacccttgcatccgacatcctgctgatggaacgcgggcagtcaaaggctgtcactggtaaggagcatctggaacggaacgactttatcattgagaagcagaaagctgaactgcaacgcatggatgcagccaaacggcacaaagaagaacagataaatcttgccgagcaggaactgaaacaggtgaaatcagaaatacgcactgacaagttaaagaagacagccaccacggcagcgacagccataactagtggagttgcttctcttttcgggagtggaaaactgaaagaactggaacgtgccaacgaaaaactgcaagacgaggtttcaaaacggaacaccaatattgaaaaattgcagagccaagtacagcagatgtcagaaacagcatatacgcaaatccacaatctcagagaaatgcacaggcaggaacttgacatgaaagaaaaagaactgtcacggctcgccagaatcatagacaaggctttcaggtggtttccgatgttcagggaaatgctgcgcatggaaaagttttgtgccatgctgggattctctaaagaaatgactgaaagtcttatagtcaaaaaagaagccctgaaatgtagcggtaaaatctattccgagcaacacaggcggaactttgatataaaggatgatattttaagggtggaaaatgaccctgacgatgaaagcaggctgaacctgacaataaacaggaagccgattgccgactggttcagggagcaatggcacaggcttagatatggagcaagagtgccgcaacaggaagaaagaaaaagtagaggattcaaattataatagaagcaatttgattagtaatctaaaagcactccgataacgattagagtgcttttagattgtttatcattaattatcaaagcaagtgcagtttaagattttactgaagtttgcattaataaagaatatactacagctgatatatgcgcaacatattgtgacgcttgtgatttatttcccttgaaatccttaacaaataccgctaaggtataactgatattattaggcagacatatataggcaacatcattgtgagctgcaagaacaccattttcattaacataacctgaacctgtcttatgcgctataacaaccccttctttatcaagaagtggagctgctatcctatctacacctgttttgcattcttttaacgtattcttaatgaaactttgtttctcatcatcgataagaccttcagtaaacaaacgattcatcaacattgcagcaccaagaggagatgtatagttagagtaagccttgttatggtcagccgacatttcctcttccgtataagctatctgaaaacttgaacgaggaatgagtgtggctataaaactatctgtttgagcgacattaaccatatccttaaacataaggttgcttgcattgttgtcactctgagtaagagtataacgcagcaaatctctcactgtcaatgatatgactggccctgaataatctttcagcataggactccaagtctttgggtcaagtttatccctatttatatttactaaggtatcaagtgaaattcctttattgtcaaagtcattacaaagagctaatgcctgatgaaccttaaacacactcatcataggataaacactcttattattgaccttaaccgtatctctgttattaacaataaccgccacaccaatttcgccaggacaagctgagacaatttgagaaatgctatcagtcaaaacatttgttaaaggaggatttgcgctatcttttgtcgctgatttatggaacaatgaaaataccaagatgaaaatgcaaactaaagctatactcaaaactacgatttgtttttttctgtttttttccatgtttatattatttatatttgtttgacgagaatatctttatttgccgacaaaggtacataactaaagtttcccacccaaataaatagatagaaaaataacagtttgtcgaattttctttgtaaattagtaatcgctaaagaactgattttaaagacaagacaacaaaactgttatggaagcaaaaatagagaaaataagtgagttatccaaacttttgagtgttaaaacccgaatgagtgatgatttatttcatctttttggcaagtttggcatcggtcacctattatctcgcctttcattggagaaacaggacggagtttcggcttcggagttgctcctctctctttgcctcttccgcattgtgggcgagagcatccatagtatatgcaaacataagatatatgagctttcaaatcatggtaagaactgtttctatcgcatgatgattcgcccacagatggattggagacgattgatgaaccactttgcactgcgttatatgtgcctattgcgtaagtatggcgaagttcctcaatcagataccactacatgtttcattatagatgacaccgtgcttgagaagagtggtgtgaggatggagggtatcagtcgtgttttcgaccatatgaaaggcagatgcgtattgggctacaagctgttactttgtgccttctttgacggcaagacaactataccctttgatttttcactacatcaagaaaaggggaagcaaggcaactgcgggctgacaagacagcaactcaaaaaggcatatcacaccaagaggaacaccggcaatcctgattataagcgctttcaagagtgtaagatgtctaagatggaagttgccatggatatgcttcgccgtggatggaagatggggttacatgcgaagtatgtgattaccgatagttggttcacctgcgagcaacttatgacatgtgttagaagcataggtaaaggggcaatgcactttgttggacttgcaaaaatgggaaagacaaaatacactatatcgggcaaaaagaaaaatgctgcagaactcattgccacctatgaacgtgaacgaggaaagaactgtcgtaaatacaaatgtcgatatattcagctcaacggcaacttaggagatatacctatcagaatcttcctcatcaagtatggtagaaactccgcatggaacgttctgctcaccacggatacaacgatgtatttcgtaaaagcctttgaagtgtatcagattagatggaacatagaggtgatgaacaaggagactaagcaatatctcggattaggaggttatcaaggttgtgactttaatggtcagatagccgacgcaacgctgtgttaccttacatataccgtcatggctttggaaaagagattcacagaatatcaaaccatgggcgaactcttttcggatatggagggtgatctcatggcactcacgttatggaagcgagttcttacctgcatcgaacgcattcttcgcattttaggagaaatacttggaatgacgccccaataccttatggctacaatcagcggaaacgacaaagagatgagcaaaatccttgtaatggctgaagcgttggaaaaatgggacgaagtatgtggatagtctgcataacttctgttaggcatgtgttaacagtcaatggatatgggggaagacagacaaaggaaaatgagaaaagttagtgttttaaggggtgggaaactttagtacataactttacggaaaaatatgtttctaaaatatataaatcagggcaaccgcatcaaaatgtcaataacctgataatcaatacttgtcgattttgtattaatagggataagtggggtggatacaaggatgagaagtttcaacaatttacaatatccccccttaaaaatgcataattttgtatttgtctgaataatagcctgttgtcaaaaatcatgtttcttaaaaaatattacccgcaagggaaatttgaattatatggggcaaaaaatcaatttttatggggtagtagtttcgatttggaccaatggtttttttaggaaaatggacaataagggagaggaggaggttgaaaactacgtttgtttcctaagctaacgttattttgatgcggttgccctgtatataaatagacagctgtggggaaaatgtggggaaaaattagataattaaaaaggctaaatgactgaaaataatcacttagccttttaatccgtacccagacccgtacttcgtaattgctgcgcccatcattcaagactgtcaaatcgggtctttcctgtcaagccaatgatgcctatgcgtatgccaaacacatggattacggatttctcgttccgtgaacaaacactttatccgcaactctgctatgtggtgtattggcttaactccatttctatgggcaacacttttgttgcagatttcaagcagcttttatcgaaatacccatcagtaagaactcgtttattaggctttcctcataattgggaacaagagcctttgtggagataaaataattgccctgtttcttaaaaagcactggggcaaaccagctccgtctttaattgtttccaataaatggattgtttcaagcccctatagaaagagaacaaaaattcctgtgtgaaaattaatctacgacaaggagcaagcaaggagcaatatcaaacaaaaatcaatacctttgcagtacatatgagatagaccaaaacaaaagtggaatatcggaaacgaatagcaaggaaaagagaagaaacgacccaagttgatgtcctttttgagttaataatcaataaaagcgttaaatcttcaccttttagaatgtgcaattaaaaataacaaccatatttctgacttattatctataaaatattgagtaataatcggttgtaaataccgaacc

SEQ ID NO: 3 - DNA, 481 bp, Bacteroides fragilis
  ref <308>: DATABASE ACCESSION NUMBER: AF172709
  ref <309>: DATABASE ENTRY DATE: 1999-08-11
  ref <313>: RELEVANT RESIDUES IN SEQ ID NO: (1)..(481)
  ref <301>: AUTHORS: Kuwahara,T., Akimoto,S., Kataoka,K., Nakayama,H., Arimochi,H. and Ohnishi,Y.
  ref <302>: TITLE: Direct Submission - Bacteriology, The University of Tokushima, School of Medecine, 3-15-15, Kuramoto-cho, Tokushima 770-8503, Japan
  ref <306>: PAGES: Direct Submission
  ref <307>: DATE: 1999-07-26
  ref <308>: DATABASE ACCESSION NUMBER: AF172709
  ref <309>: DATABASE ENTRY DATE: 1999-08-11
ggagcgatgttgaaaacgacgttcattggttctaaattgtactactggtacttgtttatttatagaatatagatcaaccatctatagcaatatagatagagataaacaagagaaagacagaagccgagtctaaaacaggacacggttgaactagtcctatagctcagttggttagagcgctacactgataatgtagaggtcggcagttcaactctgcctgggactaccaacagatagatattttatcttgtatgattgggggattagctcagctggctagagcatctgccttgcacgcagagggtcaacggttcgaatccgttattctccactccgataccgcgacctaacaggcttgcgtgtaatcaaacgatctttgacatgatgtacagaaaaaagtaaatttagtaagagctaaaagtatatatcgaaccatttggttcagatcctttttaagaaaaggagatgaatctggcgataa

SEQ ID NO: 4 - DNA, 638 bp, Bacteroides fragilis
  ref <301>: AUTHORS: Lin,Y.T., Vaneechoutte,M., Huang,A.H., Teng,L.J., Chen,H.M., Su,S.L. and Chang,T.C.
  ref <302>: TITLE: Identification of clinically important anaerobic bacteria by an oligonucleotide array
  ref <303>: JOURNAL: Journal of Clinical Microbiology
  ref <304>: VOLUME: 48
  ref <305>: ISSUE: 4
  ref <306>: PAGES: 1283-1290
  ref <307>: DATE: 2010
  ref <308>: DATABASE ACCESSION NUMBER: GQ496395
  ref <309>: DATABASE ENTRY DATE: 2010-04-05
  ref <308>: DATABASE ACCESSION NUMBER: GQ496395
  ref <309>: DATABASE ENTRY DATE: 2010-04-05
  ref <313>: RELEVANT RESIDUES IN SEQ ID NO: (1)..(638)
ccggaaggtgcggctggaacacctcctttctggagcgatgttgaaaacgacgttcattggttctaaaattgtactactggtacttgtttatttatagaatatagatcaaccatctatagcaatatagatagagataaacaagagaaaaacagaagccgagtctaaaacaggacacggttgaactagtcctatagctcagttggttagagcgctacactgataatgtagaggtcggcagttcaactctgcctgggactaccaacagatagatattttatcttgtatgattgggggattagctcagctggctagagcatctgccttgcacgcagagggtcaacggttcgaatccgttattctccactccgataccgcaacctaacatgcttgcgtgtaatcaaacgatctttgacatgatgtacagaaaaaagtaaatttagtaagagctaaaagtatatatcgaaccattgggttcatctcctttcaaaggaaatgatccctggcgataaaagaaagtaaataagggcgcatggcggatgccttggctctcggaggcgatgaaggacgtgataagctgcgataagcttcgggtaggtgcaaatgacctttgatccgaagatttccgaatgggacaaccc

(19) United States
(12) Patent Application Publication
(10) Pub. No.: US 2021/0254122 A1
(43) Pub. Date: Aug. 19, 2021

(54) SENSOR FOR INDICATING A POTENTIAL FORTHCOMING SKIN OR GASTROINTESTINAL ISSUE AND METHODS OF USING THE SAME

(71) Applicant: Kimberly-Clark Worldwide, Inc., Neenah, WI (US)

(72) Inventors: Cindy Korir-Morrison, Smyrna, GA (US); David W. Koenig, Menasha, WI (US)

(73) Assignee: Kimberly-Clark Worldwide, Inc., Neenah, WI (US)

(21) Appl. No.: 17/041,603

(22) PCT Filed: Mar. 28, 2019
(86) PCT No.: PCT/US19/24554

§ 371 Date: Sep. 25, 2020

Related U.S. Application Data
(60) Provisional application No. 62/649,778, filed on Mar. 29, 2018.

(51) Int. Cl. C12Q 1/04; C12Q 1/689

(52) U.S. Cl. CPC C12Q 1/04; C12Q 1/689

(57) ABSTRACT

A sensor for detecting and indicating a potential forthcoming skin or gastrointestinal issue includes a detection mechanism and an output mechanism. The detection mechanism is configured for detecting at least one Bacteroides virulence factor present from a sample location on a subject. The output mechanism is configured to provide an indication of a potential forthcoming skin or gastrointestinal issue based on at least one Bacteroides virulence factor being detected. Additional embodiments include a sensor having a detection mechanism configured for detecting an amount of Bacteroides and an amount of Anaerococcus from the sample location and an output mechanism configured to provide an indication of a potential forthcoming skin or gastrointestinal issue based on a Bacteroides to Bacteroides ratio of at least 0.4 being detected. The sensor may be incorporated into absorbent articles, such as diapers, training pants, adult incontinence products, female hygiene products, or wipes.

### TECHNICAL FIELD

The present disclosure relates to sensors. More specifically, the present disclosure relates to sensors that can be used to indicate potential forthcoming skin or gastrointestinal issues.

### SEQUENCE LISTING

The present applications contains a Sequence Listing which has been submitted electronically in ASCII format and is hereby incorporated by reference in entirety. The ASCII copy, created on Apr. 9, 2021, is named 65051135US10PATENT_IN_FileST25.txt, and is 23,388 bytes in size.

### BACKGROUND OF THE DISCLOSURE

Skin rashes are a relatively common episode for humans. One type of skin rash that is particularly common for those wearing absorbent articles is diaper dermatitis, commonly referred to as diaper rash. For example, it is estimated that between 50-100% of infants will have at least one episode of diaper rash before the age of 2 years. Diaper rash can be bothersome for a wearer of an absorbent article, as well as a source of frustration for a caregiver. Although there are several primary causes of diaper rash, one of the biggest factors relates to habits and practices of cleaning skin and frequency in changing an absorbent article. Inadequate cleaning and/or inadequate frequency of diaper changes can greatly contribute to the number of incidences and severity of diaper rash.

While there are various products available to treat skin rashes, such as a diaper rash, there is not believed to be any commercially available predictive technology or indicators allowing a user or caregiver to assess adequate cleaning practices or indicate an impending diaper rash episode or gastrointestinal issue.

Thus, there is a desire for a sensor that can indicate a potential forthcoming skin or gastrointestinal issue. More particularly, there is a desire for a sensor that can indicate a potential forthcoming diaper rash.

### SUMMARY OF THE DISCLOSURE

In one embodiment, a sensor can include a first detection mechanism configured for detecting at least one Baderoides virulence factor present from a sample location. The sensor can also include an output mechanism configured to provide an indication of a potential forthcoming skin or gastrointestinal issue based on at least one Baderoides virulence factor being detected.

In another embodiment, a sensor can include a detection mechanism configured for detecting an amount of Bacteroides and an amount of Anaerococcus from the sample location. The sensor can additionally include an output mechanism configured to provide an indication of a potential forthcoming skin or gastrointestinal issue based on a Bacteroides to Anaerococcus ratio of at least 0.4 being detected.

In yet another embodiment, a method for indicating a potential forthcoming skin or gastrointestinal issue in a subject can include providing a first sensor. The first sensor can include a first detection mechanism configured for detecting at least one Bacteroides virulence factor present from a sample location on the subject. The method can include applying the sensor to the subject at a primary sampling time. The method can also include indicating the potential forthcoming skin or gastrointestinal issue when at least one Bacteroides virulence factor is detected at the primary sampling time.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is a graph showing a Bacteroides population from a study of both rash prone and non-rash prone individuals both during rash and recovery from rash.

FIG. 2 is a graph showing Anaerococcus population from a study of both rash prone and non-rash prone individuals both during rash and recovery from rash.

FIG. 3 is front perspective view of an exemplary embodiment of an absorbent article, such as a diaper, in a fastened condition.

FIG. 4 is a top plan view of the absorbent article of FIG. 3 in a stretched, laid flat, unfastened condition.

FIG. 5A is a cross-section taken along line 5-5 from FIG. 4.

FIG. 5B is a cross-section similar to FIG. 5A, but showing an alternative embodiment.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards a sensor 15 configured for indicating a potential forthcoming skin or gastrointestinal issue, as well as a method for indicating such a potential issue. In a preferred embodiment, the sensor 15 can be configured for indicating a potential forthcoming diaper rash. By providing advanced notice of a potential forthcoming skin or gastrointestinal issue, a user or caregiver can modify or adapt their behavior to try to prevent, or at least lessen the severity of, any forthcoming skin or gastrointestinal issue. For example, in the context of diaper rash, early indication of a potential forthcoming diaper rash allows a caregiver or user an opportunity to adjust his/her cleaning and/or diapering regimen to prevent a forthcoming diaper rash from forming, or reducing the severity of such a diaper rash.

In preferred embodiments, the sensor 15 can be incorporated into an absorbent article 10 and form a component or a portion of a component of an absorbent article 10. In some embodiments, the sensor 15 can be incorporated into a wipe and form a component or a portion of a component of a wipe. It is contemplated, however, that the sensor 15 of the present disclosure can be a standalone item as well. Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements.

The terms “comprising”, “including” and “having” are intended to be inclusive and mean that there may be additional elements other than the listed elements. As used herein, the terminology of “first,” “second,” “third”, etc. does not designate a specified order or that items referred to using such terms must be present sequentially, but is used as a means to differentiate between different features being described in the present disclosure. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, including, but not limited to, adult fecal incontinence garments, medical garments, surgical pads and bandages, other personal care or health care garments, and the like without departing from the scope of the present disclosure.

The term “acquisition layer” refers herein to a layer capable of accepting and temporarily holding liquid body exudates to decelerate and diffuse a surge or gush of the liquid body exudates and to subsequently release the liquid body exudates therefrom into another layer or layers of the absorbent article.

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “film” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, 5 and 10 and about 15, 20 and 40. Spunbond fibers are generally not tacky when they are deposited on a collecting surface.

The term “subject” refers herein to one to which a sensor as described herein is applied. In the context of a wearable absorbent article including a sensor, the subject can be referred to as a “wearer” of the absorbent article.

The term “superabsorbent” refers herein to a water-swellable, water-insoluble organic or inorganic material capable, under the most favorable conditions, of absorbing at least about 15 times its weight and, in an embodiment, at least about 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “thermoplastic” refers herein to a material which softens and which can be shaped when exposed to heat and which substantially returns to a non-softened condition when cooled.

The term “user” or “caregiver” refers herein to one who applies and/or reviews a sensor applied to a subject, such as, but not limited to, a sensor included in a diaper, diaper pant, training pant, youth pant, incontinent product, wipe or other absorbent article applied to or about a subject. A user and a subject (e.g., wearer) can be one and the same person.

Sensor:

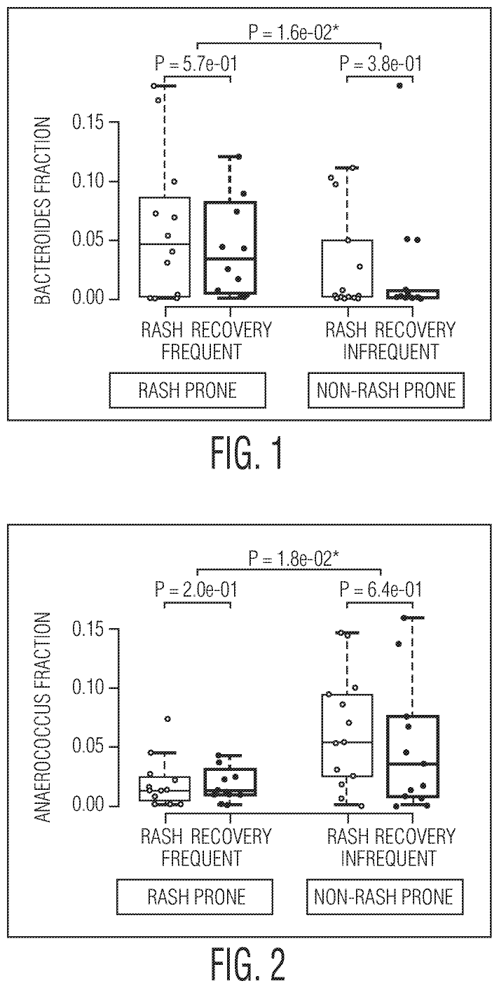

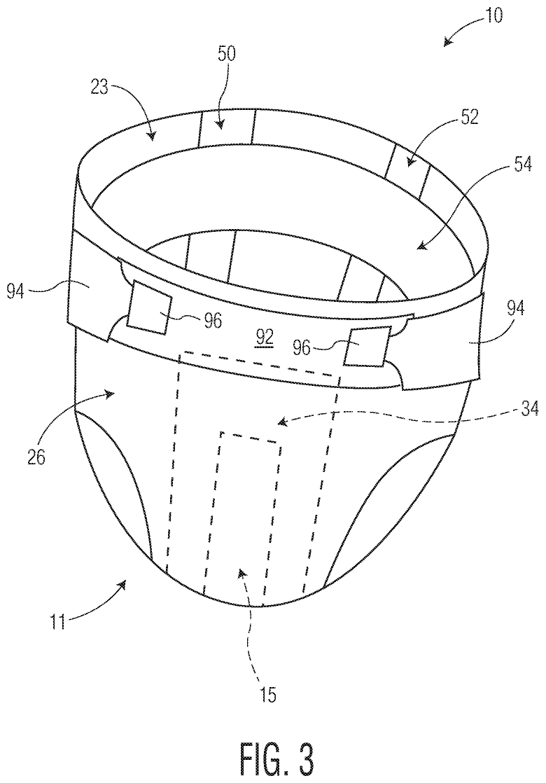

The sensor 15 and associated methods described herein can be configured to help detect and indicate a potential forthcoming skin or gastrointestinal issue. In some embodiments, the potential forthcoming skin issue could be diaper rash. In that regard, a study was conducted that included twenty-five infants ranging in age of nine to twelve months. In recruiting for the study, the infants were selected as those that were presenting with mild to moderate diaper rash symptoms confirmed by a pediatrician. In the study, swabs were taken from the area exhibiting the diaper rash during the rash as well as after recovery on the subjects. The paired swabs from the diaper rash and recovery states were used for 16S rRNA sequencing to obtain microbial community profiles. Subjects were classified as either rash prone (experienced four or more episodes of rash in the first eight to twelve months of their life) or non-rash prone (experienced one or less episodes of rash in the first eight to twelve months of their life). Tables 1 and 2 provide a summary of the mean of the results of the community profiling of Bacteroides and Anaerococcus, respectively, from the 16S rRNA sequencing. Additionally, FIGS. 1 and 2 illustrate the results of the 16S rRNA sequencing for Bacteroides and Anaerococcus, respectively, in graph form.

TABLE 1
Bacteroides community profile
Bacteroides (B) Abundance (mean)

| | Rash Prone Infant | Non-Rash Prone Infant |
|---|---|---|
| Rash | 0.060 | 0.031 |
| No Rash (Recovery) | 0.054 | 0.023 |

TABLE 2
Anaerococcus community profile
Anaerococcus (A) Abundance (mean)

| | Rash Prone Infant | Non-Rash Prone Infant |
|---|---|---|
| Rash | 0.020 | 0.064 |
| No Rash (Recovery) | 0.034 | 0.069 |

As shown in Table 1 and FIG. 1. the results showed that the community profile of Bacteroides was higher in the rash state than the recovery state in each of the respective classifications of rash prone subjects and non-rash prone subjects. Additionally, the results indicated an association between Bacteroides and subjects that experience frequent diaper rash in that the Bacteroides fraction in the microbial community was higher for subjects that frequently experienced diaper rash (“rash prone”) as compared to subjects that infrequently experienced diaper rash (“non-rash prone”). Table 1 and FIG. 1 also demonstrate that the Bacteroides fraction in the community profile exhibited more change between rash and recovery states in non-rash prone subjects as compared to rash prone subjects. Not to be bound by theory, but it is believed that inadequate cleaning practices or prolonged changing intervals may have been occurring in the rash prone subjects even in the recovery state as the Bacteroides community fraction remained relatively constant as compared to the rash state in such subjects. Additionally, Bacteroides may be present on the skin due to gastrointestinal inflammation events that lead to the increase in Bacteroides abundance and/or associated virulence factors found in fecal material.

As demonstrated in Table 2 and in FIG. 2, the Anaerococcus community profile exhibited a lower fraction for rash prone subjects (in both rash and recovery states) as compared to the fraction of Anaerococcus in non-rash prone subjects. The amount of Anaerococcus in non-rash prone subjects was relatively consistent between the recovery state and the rash state. Similarly, the amount of Anaerococcus in rash prone subjects was relatively consistent between the recovery state as compared to the rash state.

Table 3 shows the ratio of the community profile of Bacteroides to the community profile of Anaerococcus (BA ratio) for comparative community profiles of Tables 1 and 2 and as illustrated in FIGS. 1 and 2.

TABLE 3
Bacteroides:Anaerococcus Community Profile Ratios
Bacteroides to Anaerococcus (B:A) Ratio

| | Rash Prone Infant | Non-Rash Prone Infant |
|---|---|---|
| Rash | 3.0 | 0.5 |
| No Rash (Recovery) | 1.6 | 0.3 |

In reviewing this comparative data, it was discovered that the ratio of Bacteroides to Anaerococcus (BA ratio) can be helpful in determining a potential for a forthcoming skin rash or gastrointestinal issue. As Anaerococcus is a normal skin flora, it may be expected to be found on the skin. In that Baderoides can be found in the gastrointestinal tract, normal fecal material may be expected to include Bacteroides. If there are good hygiene practices, the fecal material should be removed reducing or eliminating the Bacteroides found on the skin. For example, it is believed that a BA ratio of less than or equal to 0.3 can indicate a low risk for an oncoming skin rash and/or gastrointestinal issue. It is believed that a B:A ratio of 0.4 to 1.0 can indicate a slight risk for an oncoming skin rash and/or gastrointestinal issue and that cleaning habits may need to be improved, with some of the Baderoides remaining on the skin. A B:A ratio of 1.0 to 2.0 can indicate a risk for an oncoming skin rash and/or gastrointestinal issue and that cleaning habits may need significant improvement. Furthermore, a BA ratio of 2.0-3.0 can indicate a moderate risk for an oncoming skin rash and a possible gastrointestinal issue. Lastly, a B:A ratio of greater than 3.0 can indicate a high risk for an oncoming skin rash and a probable gastrointestinal issue. Even with significant cleaning, a high amount of Bacteroides may be left on the skin in such a circumstance with a high risk for an oncoming rash and/or gastrointestinal issue.

The sensor 15 of the present disclosure may be configured to detect and identify at least one Baderoides virulence factor to provide information as to the presence and/or approximate amount of Baderoides from a sample area of a subject, and in turn, a potential for a forthcoming skin or gastrointestinal issue. In some embodiments, the sensor 15 can be configured to detect two or more Baderoides virulence factors. In some embodiments, the sensor 15 can be configured to detect three or more Baderoides virulence factors. The sensor 15 of the present disclosure includes at least one detection mechanism and at least one output mechanism. In the context of diaper rash, the sample area of a subject can be skin in the genital region, and thus, the sensor 15 can be applied on or near that sample area on a subject.

Multiple platforms are possible to detect Baderoides virulence factors, and thus, detection mechanisms can be varied. For example, platforms include antibody linked assays targeting unique cell wall proteins and biochemical structures. Other means that are possible include utilizing lectin, nucleic acid, and phage based binding targets.

All of these potential configurations for detection mechanisms can allow for output mechanisms of various types to provide various signals to a user or caregiver to identify the presence and/or amount of one or more Bacteroides virulence factors, and in turn, an indication of the potential for a forthcoming skin or gastrointestinal issue. In some embodiments, the output mechanism of the sensor 15 may need to be viewed by a user to determine if it is indicating a potential forthcoming skin or gastrointestinal issue. In other embodiments, the output mechanism may communicate the output to a user such that the user need not be viewing the sensor 15 itself, for example, through an audible or tactile signal, or through communication on other aspects of the absorbent article 10 or remote devices. In some embodiments, an output mechanism can provide spectral signals in either visible or non-visible means, such as fluorescent, visible, IR, and photon emitting, to identify the presence and/or amount of one or more Bacteroides virulence factors. Additionally, it is possible to configure the output mechanism to generate other signals, such as changes in impedance/conductivity, as well as generation of electrical out-put, to identify the presence and/or amount of one or more Baderoides virulence factors. As a result, an output mechanism can be configured to notify a user or caregiver of the presence and/or amount of one or more Baderoides virulence factors as an indication of a potential forthcoming skin or gastrointestinal issue when a detection mechanism detects at least one Baderoides virulence factor.

Baderoides virulence factors that are possible for detection can include Bacteroides structural targets, human produced targets associated with Bacteroides, Bacteroides produced targets, and Baderoides genomic elements targets. Bacteroides structural targets can include, but are not limited to, capsular polysaccharides, zwitterionic polysaccharides, lipopolysaccharides, pili fimbriae, and Omp 200. Human produced targets can include, but are not limited to, Regllly and antibiotic Paneth cell protein (Ang4). Baderoides produced targets can include, but are not limited to, Hyalunidase and chonodroitin sulfatase, hemolysins (HlyA and HlyB), Neuraminidase, Enterotoxin (BFT), Endotoxin (LPS), and Bacteriocin protein. Bacteroides genomic elements targets can include, but are not limited to, a sequence including sequence NO. 1, sequence NO. 2, sequence NO. 3, and sequence NO. 4 in the Sequence Listing.

The sensor 15 can be configured such that the detection mechanism can be configured to detect the presence of one or multiple Bacteroides virulence factors. In one preferred embodiment, a detection mechanism of the sensor 15 can be configured to detect Enterotoxin (BFT). Detection of Enterotoxin (BFT) can indicate a high risk of a potential oncoming skin or gastrointestinal issue, and the output mechanism can be configured to indicate as such. The sensor 15 can be configured such that if any Baderoides virulence factors other than Enterotoxin (BFT) is detected, the output mechanism can indicate a risk of a potential oncoming skin or gastrointestinal issue. The sensor 15 can be also configured such that if two Bacteroides virulence factors other than Enterotoxin (BFT) are detected, the output mechanism can indicate a medium risk of a potential oncoming skin or gastrointestinal issue. In some embodiments, the sensor 15 can be further configured such that if three Baderoides virulence factors other than Enterotoxin (BFT) are detected, the output mechanism can indicate a high risk of a potential oncoming skin or gastrointestinal issue.

In other embodiments, the sensor 15 can be configured such that the output mechanism is configured to provide the indication of a potential forthcoming skin or gastrointestinal issue when a detection mechanism detects at least a specified threshold level of at least one Baderoides virulence factor. In one embodiment, the specified threshold level of at least one Baderoides virulence factor can be an amount of at least 1.5 times of a lower detection limit of an assay that is being used as the detection mechanism. In some embodiments, the threshold value of a Baderoides virulence factor can be about 100 pg/mL.

In some embodiments, a sensor 15 of the present disclosure can be configured to detect an amount of Bacteroides in comparison to an amount of Anaerococcus in a sample area of a subject and indicate a potential forthcoming skin or gastrointestinal issue based on a ratio Baderoides to Anaerococcus. A detection mechanism configured to detect an amount Bacteroides and an amount of Anaerococcus can be provided in a sensor 15 either as an additional detection mechanism or as a stand-alone detection mechanism in the sensor 15. As discussed above with respect to Table 3, an output mechanism can be configured to provide an indication of a potential forthcoming skin or gastrointestinal issue when a detection mechanism detects the amount of Bacteroides in comparison to an amount of Anaerococcus to be in a ratio of at least 0.4. In some embodiments, an output mechanism can be configured to provide an indication of a potential forthcoming skin or gastrointestinal issue when a detection mechanism detects the amount of Bacteroides in comparison to an amount of Anaerococcus to be in a ratio of at least 1.0, or at least 2.0, or at least 3.0.

In some embodiments, the sensor 15 can include a first detection mechanism configured to detect at least one Bacteroides virulence factor present from a sample location, a second detection mechanism configured to detect an amount of Bacteroides and an amount of Anaerococcus from the sample location, and an output mechanism configured to provide an indication of a potential forthcoming skin or gastrointestinal issue when the first detection mechanism detects at least one Bacteroides virulence factor and the second detection mechanism detects a ratio of Bacteroides to Anaerococcus is present of at least 0.4, or at least 1.0, or at least 2.0, or at least 3.0.

In some embodiments, the sensor 15 can include a first detection mechanism configured to detect two or more Bacteroides virulence factors present from a sample location and a second detection mechanism configured to detect an amount of Bacteroides and an amount of Anaerococcus from the sample location. The sensor 15 can also include an output mechanism configured to provide an indication of a potential forthcoming skin or gastrointestinal issue when the first detection mechanism detects at least one Bacteroides virulence factor and the second detection mechanism detects a ratio of Bacteroides to Anaerococcus is present of at least 0.4, or at least 1.0, or at least 2.0, or at least 3.0. Such a sensor 15 can be configured to indicate a potential forthcoming skin or gastrointestinal issue at a lower BA ratio when detecting two or more Bacteroides virulence factors as compared to when detecting a higher B:A ratio and only one Bacteroides virulence factor. As but one example, a sensor 15 could be configured to indicate a potential forthcoming skin or gastrointestinal issue based on the detection of two Bacteroides virulence factors and a B:A ratio of 1.0 or greater, but be configured to only indicate a potential forthcoming skin or gastrointestinal issue based on the detection of only one Bacteroides virulence factor and a B:A ratio of 2.0 or greater. The output mechanism can be configured to provide an indication of such a potential forthcoming skin or gastrointestinal issue through various signals and/or technologies as previously described.

The sensors 15 as described herein can also be utilized in a method to indicate a potential forthcoming skin or gastrointestinal issue in a subject when an increased threshold occurs for one or more Bacteroides virulence factors and/or an increased threshold in Bacteroides to Anaerococcus ratio. For example, a sensor 15 can be configured to indicate a potential forthcoming skin or gastrointestinal issue in a subject when an increased threshold occurs in at least one type Bacteroides virulence factor as detected over the course of time from a primary sampling time to a secondary sampling time. In one embodiment, the method can include providing a first sensor 15 as described above including a first detection mechanism that is configured for detecting at least one Bacteroides virulence factor present from a sample location on the subject. The sensor 15 can also include a first output mechanism. The sensor 15 can be applied to the subject. The first output mechanism can be configured to provide an indication of an amount of the at least one Bacteroides virulence factor after detection by the first detection mechanism at a primary sampling time. The method can further include later providing a separate sensor 15 to determine if the amount of Baderoides virulence factor has increased from the primary sampling time. For purposes of this discussion, the later sensor 15 is referred to as the third sensor 15. The third sensor 15 can be configured similar to the first sensor 15 described above and can detect an amount of the at least one Bacteroides virulence factor present on the sample location on the subject at a secondary sampling time. The third sensor 15 can include a third output mechanism configured to provide an indication of an amount of the at least one Bacteroides virulence factor at the secondary sampling time. The third sensor 15 can be applied to the subject after the first sensor 15 has been removed from the subject after the primary sampling time. The method can include indicating the potential forthcoming skin or gastrointestinal issue when the amount of the at least one Bacteroides virulence factor detected by the third detection mechanism at the secondary sampling time is greater than the amount of the at least one Bacteroides virulence factor detected by the first detection mechanism at the primary sampling time.

As an example, the first sensor 15 could be configured to provide a qualitative indication of the amount of Bacteroides virulence factor through the first output mechanism at the primary sampling time, such as through an indication of a green color, indicating a very low to no presence of a particular Bacteroides virulence factor. The third sensor 15 can provide a qualitative indication of the amount of a Bacteroides virulence factor through the third output mechanism, such as through an indication of a red color, indicating a higher amount, or presence, of the particular Bacteroides virulence factor from the sample area at the secondary sampling time. This increase in threshold of the Bacteroides virulence factor over time can indicate to a user or caregiver that the subject may have a potential forthcoming skin or gastrointestinal issue. Additionally, it is contemplated that a sensor 15 could be configured to monitor the threshold increase or decrease for two or more Bacteroides virulence factors. Of course, it is contemplated that the sensor 15 can be configured to analyze the data from the detection mechanism of the first sensor 15 and from the third sensor 15 to indicate to the user or caregiver that the subject may have a potential forthcoming skin or gastrointestinal issue through various other signals and means, as described above in the various exemplary output mechanisms.

Alternatively, a sensor 15 can be configured such that the sensors are configured to detect the Bacteroides to Anaerococcus ratio from a primary sampling time to a secondary sampling time and indicate to the user or caregiver that the subject may have a potential forthcoming skin or gastrointestinal issue if the Bacteroides to Anaerococcus ratio increased from the secondary sampling time as compared to the primary sampling time.

In another embodiment, the method can include sensors 15 to indicate a potential forthcoming skin or gastrointestinal issue in a subject when an increase in the ratio of Baderoides to Anaerococcus is detected over time from a primary sampling time to a secondary sampling time. For purposes herein, the sensors 15 for measuring a ratio of Baderoides to Anaerococcus will be referred to as a second sensor 15 and a fourth sensor 15, however, these two sensors 15 could be utilized in their own methodology independent of any sensors detecting Bacteroides virulence factors as described above. The second sensor 15 can be as described above and include a second detection mechanism that is configured to detect an amount of Bacteroides and an amount of Anaerococcus. The second sensor 15 can include a second output mechanism to indicate the ratio of Bacteroides to Anaerococcus at a primary sampling time. The fourth sensor 15 can include a fourth detection mechanism and be configured to detect an amount of Bacteroides and an amount of Anaerococcus and a fourth output mechanism to indicate a ratio of Bacteroides to Anaerococcus at a secondary sampling time. As such, in some embodiments, the method can include indicating the potential forthcoming skin or gastrointestinal issue when the ratio of Bacteroides to Anaerococcus at the secondary sampling time is greater than the ratio of Bacteroides to Anaerococcus at the primary sampling time. In some embodiments, the method can include indicating the potential forthcoming skin or gastrointestinal issue when the amount of the at least one Bacteroides virulence factor detected by the third detection mechanism at the secondary sampling time is greater than the amount of the at least one Bacteroides virulence factor detected by the first detection mechanism at the primary sampling time and when the ratio of Bacteroides to Anaerococcus as detected by the fourth detection mechanism at the secondary sampling time is greater than the ratio of Bacteroides to Anaerococcus as detected by the second detection mechanism at the primary sampling time.

In some embodiments, the second sensor 15 configured for detecting an amount of Bacteroides and an amount of Anaerococcus and the first sensor 15 configured for detecting an amount of at least one Bacteroides virulence factor can be applied to the subject at the same time and can be disposed on or form part of the same substrate or article. In one embodiment, this can be accomplished by having the first and second sensors 15 incorporated in the same article, such as the same absorbent article 10. In some embodiments, the fourth sensor 15 configured for detecting an amount of Bacteroides and an amount of Anaerococcus and the third sensor 15 configured for detecting an amount of at least one Bacteroides virulence factor can be applied to the subject at the same time and be disposed on or form part of the same substrate or article. Similarly, this can be accomplished by having the third and fourth sensors 15 incorporated in the same article, such as the same absorbent article 10.

The sensor(s) 15 as described herein can provide the advantage of providing an early indication of a potential forthcoming skin or gastrointestinal issue on the subject before it is substantial and/or severe enough to be visually apparent and/or in which the subject feels more significant discomfort. By providing such early notification, a user or caregiver can modify or adapt their behavior to try to prevent, or at least lessen the severity of, any forthcoming skin or gastrointestinal issue. For example, in the context of diaper rash, early indication of a potential forthcoming diaper rash allows a caregiver or user an opportunity to adjust his/her cleaning and/or diapering regimen to prevent a diaper rash from forming, or reducing the severity of such a forthcoming diaper rash.

Absorbent Article:

As previously noted, the sensor 15 as described herein can form a component of, or be disposed on or within a component of an absorbent article 10. This is particularly beneficial in circumstances in which the sensor 15 is being used to provide early indication of an oncoming diaper rash.

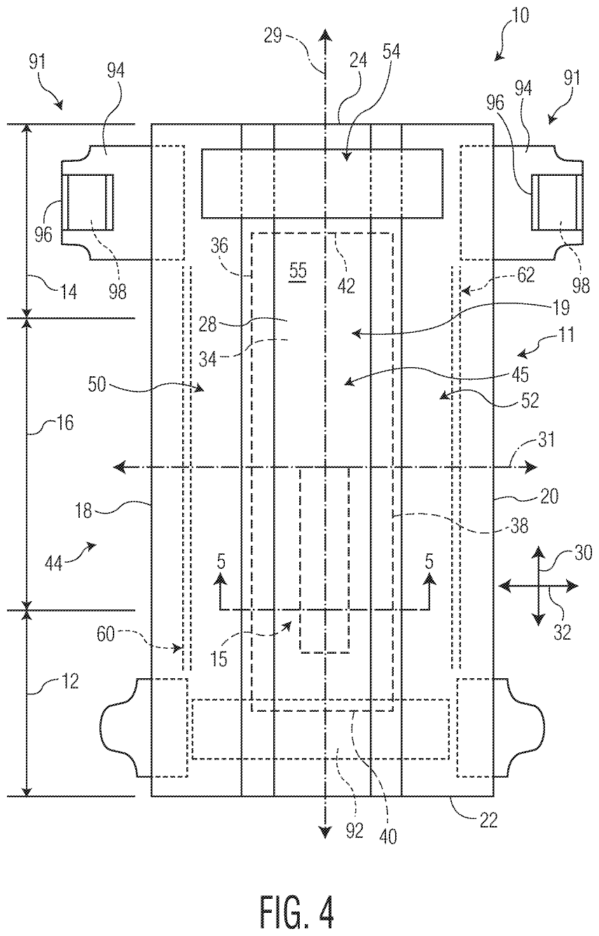

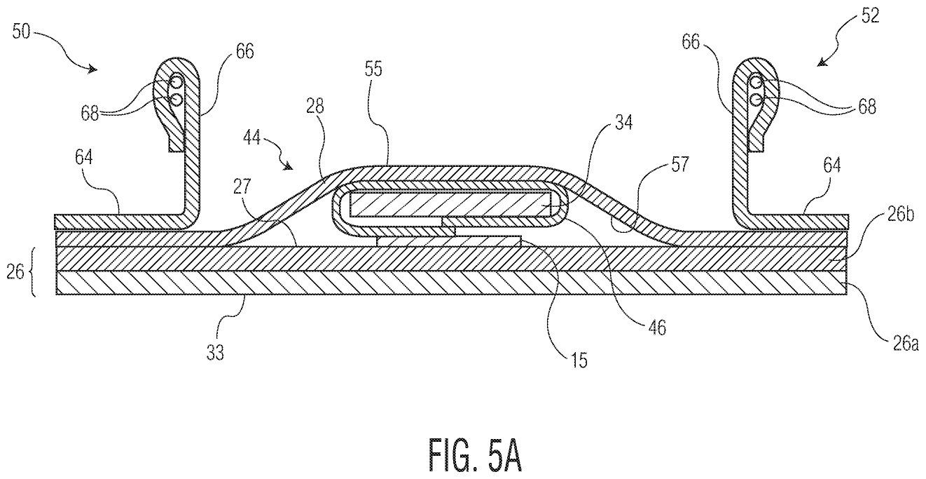

Referring to FIGS. 3 and 4, a non-limiting illustration of an absorbent article 10, for example, a diaper, is illustrated. Other embodiments of the absorbent article could include training pants, youth pants, adult incontinence garments, and feminine hygiene articles. While the embodiments and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross direction manufacturing of a product, without departing from the spirit and scope of the disclosure.

The absorbent article 10 illustrated in FIGS. 3 and 4 can include a chassis 11. The absorbent article 10 can include a front waist region 12, a rear waist region 14, and a crotch region 16 disposed between the front waist region 12 and the rear waist region 14 and interconnecting the front and rear waist regions, 12, 14, respectively. The front waist region 12 can be referred to as the front end region, the rear waist region 14 can be referred to as the rear end region, and the crotch region 16 can be referred to as the intermediate region.

As illustrated in FIG. 4, the absorbent article 10 can have a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIG. 4. In other embodiments, the longitudinal side edges 18, 20 can be curved between the front waist edge 22 and the rear waist edge 24.

The front waist region 12 can include the portion of the absorbent article 10 that, when worn, is positioned at least in part on the front of the wearer while the rear waist region 14 can include the portion of the absorbent article 10 that, when worn, is positioned at least in part on the back of the wearer. The crotch region 16 of the absorbent article 10 can include the portion of the absorbent article 10 that, when worn, is positioned between the legs of the wearer and can partially cover the lower torso of the wearer. The waist edges, 22 and 24, of the absorbent article 10 are configured to encirle the waist of the wearer and together define a central waist opening 23 (as labeled in FIG. 3) for the waist of the wearer. Portions of the longitudinal side edges 18, 20 in the crotch region 16 can generally define leg openings for the legs of the wearer when the absorbent article 10 is worn.

The absorbent article 10 can include an outer cover 26 and a bodyside liner 28. The outer cover 26 and the bodyside liner 28 can form a portion of the chassis 11. In an embodiment, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 can define a length in a longitudinal direction 30, and a width in the lateral direction 32, which, in the illustrated embodiment, can coincide with the length and width of the absorbent article 10. As illustrated in FIG. 4, the absorbent article 10 can have a longitudinal axis 29 extending in the longitudinal direction 30 and a lateral axis 31 extending in the lateral direction 32.

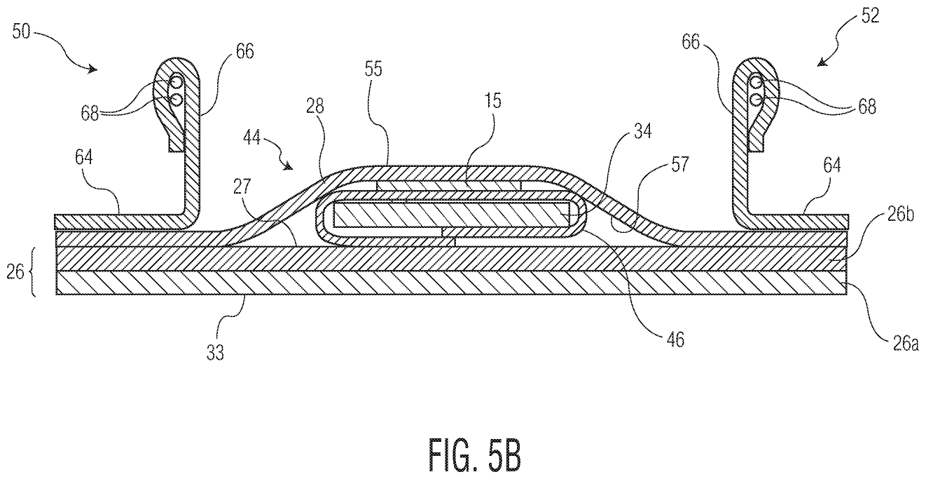

The chassis 11 can include an absorbent body 34. The absorbent body 34 can be disposed between the outer cover 26 and the bodyside liner 28. The absorbent body 34 can have a pair longitudinal side edges, 36 and 38, which, in an embodiment, can form portions of the longitudinal side edges, 18 and 20, respectively, of the absorbent article 10. The absorbent body 34 can have a first end edge 40 that is opposite a second end edge 42, respectively, which, in an embodiment, can form portions of the waist edges, 22 and 24, respectively, of the absorbent article 10. In some embodiments, the first end edge 40 can be in the front waist region 12 and can be referred to as a front end edge 40 of the absorbent body 34. In some embodiments, the second end edge 42 can be in the rear waist region 14 and can be referred to as a rear end edge 42 of the absorbent body 34. In an embodiment, the absorbent body 34 can have a length and width that are the same as or less than the length and width of the absorbent article 10. The bodyside liner 28, the outer cover 26, and the absorbent body 34 can form part of an absorbent assembly 44. The absorbent assembly 44 can also include a fluid transfer layer 46 (shown in FIGS. 5A and 5B) and a fluid acquisition layer 48 (shown in FIGS. 5A and 5B) between the bodyside liner 28 and the absorbent body 34. In some embodiments, if a fluid transfer layer 46 is present, the acquisition layer 48 can be between the bodyside liner 28 and the fluid transfer layer 46 as is known in the art. The absorbent assembly 44 can also include a spacer layer (not shown) disposed between the absorbent body 34 and the outer cover 26 as is known in the art. The absorbent assembly 44 can include other components in some embodiments. It is also contemplated that some embodiments may not include a fluid transfer layer 46, and/or an acquisition layer 48, and/or a spacer layer.

The absorbent article 10 can be configured to contain and/or absorb liquid, solid, and semi-solid body exudates discharged from the wearer. In some embodiments, a pair of containment flaps 50, 52 can be configured to provide a barrier to the lateral flow of body exudates. In some embodiments, the absorbent article 10 can include a waist containment member 54. The waist containment member 54 can be disposed in the rear waist region 14 of the absorbent article 10. Although not depicted herein, it is contemplated that the waist containment member 54 can be additionally or alternatively disposed in the front waist region 12 of the absorbent article 10.

The absorbent article 10 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10. The leg elastic members 60, 62 can be parallel to the longitudinal axis 29 as shown in FIG. 4 or can be curved as is known in the art. The leg elastic members 60, 62 can provide elasticized leg cuffs.

Additional details regarding each of these elements of the absorbent article 10 including a sensor 15 described herein can be found below and with reference to the FIGS. 3 through 5B.

Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two layer construction, including an outer layer 26a and an inner layer 26b (each shown in FIGS. 5A and 5B) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer 26a of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer 26a of an outer cover 26 can be a 20 gsm spunbond polypropylene non-woven web. The outer layer 26a may also be constructed of the same materials from which the bodyside liner 28 can be constructed as described herein.

The liquid impermeable inner layer 26b of the outer cover 26 (or the liquid impermeable outer cover 26 where the outer cover 26 is of a single-layer construction) can be either vapor permeable (i.e., “breathable”) or vapor impermeable. The liquid impermeable inner layer 26b (or the liquid impermeable outer cover 26 where the outer cover 26 is of a single-layer construction) can be manufactured from a thin plastic film. The liquid impermeable inner layer 26b (or the liquid impermeable outer cover 26 where the outer cover 26 is of a single-layer construction) can inhibit liquid body exudates from leaking out of the absorbent article 10 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

Absorbent Body:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10. The absorbent body 34 can have a length and width that can be less than or equal to the length and width of the absorbent article 10.

The absorbent body 34 includes absorbent material. In an embodiment, the absorbent body 34 can be composed of a web material of hydrophilic fibers, cellulosic fibers (e.g., wood pulp fibers), natural fibers, synthetic fibers, woven or nonwoven sheets, scrim netting or other stabilizing structures, superabsorbent material, binder materials, surfactants, selected hydrophobic and hydrophilic materials, pigments, lotions, odor control agents or the like, as well as combinations thereof. In an embodiment, the absorbent body 34 can be a matrix of cellulosic fluff and superabsorbent material. In an embodiment, the absorbent body 34 may be constructed of a single layer of materials, or in the alternative, may be constructed of two or more layers of materials.

Various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber. Suitable superabsorbent materials can be selected from natural, synthetic, and modified natural polymers and materials. The superabsorbent materials can be inorganic materials, such as silica gels, or organic compounds, such as cross-linked polymers. In an embodiment, the absorbent body 34 can be free of superabsorbent material.

If a spacer layer is present, the absorbent body 34 can be disposed on the spacer layer and superposed over the outer cover 26. The spacer layer can be bonded to the outer cover 26, for example, by adhesive. In some embodiments, a spacer layer may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer 46 and/or a spacer layer, can be positioned between the absorbent body 34 and the outer cover 26. The absorbent body 34 can be bonded to the fluid transfer layer 46 and/or the spacer layer.

Bodyside Liner:

The bodyside liner 28 of the absorbent article 10 can overlay the absorbent body 34 and the outer cover 26 and can be configured to receive insults of exudates from the wearer and can isolate the wearer's skin from liquid waste retained by the absorbent body 34. The bodyside liner 28 can form at least a part of the body facing surface 19 of the chassis 11.

In various embodiments, a fluid transfer layer 46 can be positioned between the bodyside liner 28 and the absorbent body 34 (as shown in FIGS. 5A and 5B). In various embodiments, an acquisition layer 48 can be positioned between the bodyside liner 28 and the absorbent body 34 or a fluid transfer layer 46, if present (as shown in FIGS. 5A and 5B). In various embodiments, the bodyside liner 28 can be bonded to the acquisition layer 48, or to the fluid transfer layer 46 if no acquisition layer 48 is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer 46, if present, and/or an acquisition layer 48, if present, and/or a spacer layer, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. In some embodiments, the bodyside liner 28 and the outer cover 26 may be of the same dimensions in width and length. In some embodiments, however, the bodyside liner 28 may be narrower than the outer cover 26 and/or shorter than the outer cover 26. In some embodiments, the length of the bodyside liner 28 can range from 50%-100% of the length of the absorbent article 10 as measured in a direction parallel to the longitudinal axis 29. In some embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material.

The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentangled. The projection layer can include hollow projections, such as those disclosed in U.S. Pat. No. 9,474,660 invented by Kirby, Scott S. C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or 12 to about 15 or 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10. In other aspects, the bodyside liner 28 can be stretchable, and more suitably elastic, in both the lateral and the longitudinal directions 32, 30, respectively.

Containment Flaps:

In an embodiment, the absorbent article 10 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10 through the crotch region 16 to the rear waist region 14 of the absorbent article 10. In some embodiments, the containment flaps 50, 52 can extend in a direction substantially parallel to the longitudinal axis 29 of the absorbent article 10, however, in other embodiments, the containment flaps 50, 52 can be curved, as is known in the art.

In embodiments where the containment flaps 50, 52 are coupled to the chassis 11, the containment flaps 50, 52 can be bonded to the bodyside liner 28, the outer cover 26, or another layer, such as a spacer layer, if present, with a barrier adhesive, as is known in the art. Of course, the containment flaps 50, 52 can be bonded to other components of the chassis 11 and can be bonded with other suitable means other than a barrier adhesive. For example, the containment flaps 50, 52 can be bonded to the bodyside liner 28, the outer cover 26, or another layer with pressure bonding, thermal bonding, or ultrasonic bonding. The containment flaps 50, 52 can be constructed of a fibrous material which can be similar to the material forming the bodyside liner 28. Other conventional materials, such as polymer films, can also be employed.

As illustrated in FIGS. 5A and 5B, the containment flaps 50, 52 can each include a base portion 64 and a projection portion 66. The base portion 64 can be bonded to the chassis 11, for example, to the bodyside liner 28 or the outer cover 26 as mentioned above. The base portion 64 could be bonded to other components of the chassis 11. The projection portion 66 can be configured to extend away from the body facing surface 19 of the chassis 11 (such as the body facing surface 55 of the bodyside liner 28) at least in the crotch region 16 when the absorbent article 10 is in a relaxed configuration, as illustrated in FIGS. 5A and 5B.

It is contemplated that the containment flaps 50, 52 can be of various configurations and shapes, and can be constructed by various methods. For example, the containment flaps 50, 52 of FIGS. 4-5B depict a vertical containment flap 50, 52 that can be tacked down in both the front and rear waist regions 12,14 where the projection portion 66 of each containment flap 50, 52 is tacked down to the bodyside liner 28 towards the longitudinal axis 29 of the absorbent article 10. However, it is contemplated that the containment flaps 50, 52 can be tacked down where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. patent application Ser. No. 13/900,134 by Robert L. Popp et al., which published as U.S. Patent Application Publication 201410350504. Such a configuration can also include the projection portions 66 being tacked down in either or both of the front and rear waist regions 12, 14, respectively. Of course, other configurations of containment flaps 50, 52 can be used in the absorbent article 10 and still remain within the scope of this disclosure.

The containment flaps 50, 52 can include one or more flap elastic members 68, such as the two flap elastic strands depicted in FIGS. 5A and 5B. Suitable elastic materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastic properties itself.

The flap elastic members 68, as illustrated in FIG. 4, can have two strands of elastomeric material extending longitudinally in the projection portion 66 of the containment flaps 50, 52, in generally parallel, spaced relation with each other. The flap elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, when the absorbent article 10 is in a relaxed configuration. Such an upright orientation of the projection portion 66 of containment flap 50 and the projection portion 66 of containment flap 52 is illustrated in FIGS. 5A and 5B, where the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, about 110% to about 350%. In one embodiment, the elastic members 68 can be coated with adhesive while elongated to a specified length prior to attaching to the elastic members 68 to the containment flaps 50, 52. In this exemplary method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 when the absorbent article 10 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 55 of the bodyside liner 28), as depicted in FIGS. 5A and 5B.

Leg Elastics:

Leg elastic members 60, 62 can be secured to the outer cover 26, such as by being bonded thereto by laminate adhesive, generally laterally inward of the longitudinal side edges, 18 and 20, of the absorbent article 10. The leg elastic members 60, 62 can form elasticized leg cuffs that further help to contain body exudates. In an embodiment, the leg elastic members 60, 62 may be disposed between inner 26b and outer layers 26a of the outer cover 26 or between other layers of the absorbent article 10, for example, between the base portion 64 of each containment flap 50, 52 and the bodyside liner 28, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein in FIG. 4 each include two elastic strands. A wide variety of elastic materials may be used for the leg elastic members 60, 62. Suitable elastic materials can include sheets, strands or ribbons of natural rubber, synthetic rubber, or thermoplastic elastomeric materials. The elastic materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10 without departing from the scope of this disclosure.

Waist Containment Member:

In an embodiment, the absorbent article 10 can have one or more waist containment members 54. FIGS. 3 and 4 illustrate a preferred embodiment of a waist containment member 54 on an absorbent article 10, such as a diaper where the waist containment member 54 can be disposed in the rear waist region 14. In some embodiments, the waist containment member 54 can be disposed in the front waist region 12. The waist containment member 54 can be disposed on the body facing surface 45 of the absorbent assembly 44. In some embodiments, such as in embodiments illustrated in FIGS. 3 and 4, the waist containment member 54 can be disposed on the body facing surface 55 of the bodyside liner 28. The waist containment member 54 can be coupled to the chassis 11 such that a portion of the waist containment member 54 is free to move with respect to the chassis 11 and can form a pocket to help contain body exudates.

The waist containment member 54 can be comprised of a variety of materials. In a preferred embodiment, the waist containment member 54 can be comprised of a spunbond-meltblown-spunbond (“SMS”) material. However it is contemplated that the waist containment member 54 can be comprised of other materials including, but not limited to, a spunbond-film-spunbond (“SFS”), a bonded carded web (“BCW”), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. In some embodiments, the waist containment member 54 can include an elastic material to provide additional fit and containment properties to the absorbent article 10. In such an embodiment, suitable elastic materials can include, but are not limited to, sheets, strands or ribbons of natural rubber, synthetic rubber, or thermoplastic elastomeric polymers. The elastic materials can be stretched and bonded to a substrate, bonded to a gathered substrate, or bonded to a substrate and then elasticized or shrunk, for example, with the application of heat, such that elastic retractive forces are imparted to the substrate. It is to be understood, however, that the waist containment member 54 may be omitted from the absorbent article without departing from the scope of this disclosure.

Fastening System:

In an embodiment, the absorbent article 10 can include a fastening system. The fastening system can include one or more back fasteners 91 and one or more front fasteners 92. The embodiments shown in FIGS. 3 and 4 depict an embodiment with one front fastener 92. Portions of the fastening system may be included in the front waist region 12, rear waist region 14, or both.

The fastening system can be configured to secure the absorbent article 10 about the waist of the wearer in a fastened condition as shown in FIG. 3 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 4.

Sensor:

As discussed above, a sensor 15 of the present disclosure can be implemented in an absorbent article 10, such as a diaper as described above and illustrated in FIGS. 3-5B. As illustrated in FIGS. 5A and 5B, the sensor 15 can be disposed between a body-facing surface 55 of the bodyside liner 28 and a garment facing surface 33 of the outer cover 26. As depicted in FIG. 5A, the sensor 15 can be disposed between the body facing surface 27 of the film layer 26b of the outer cover 26 and the absorbent body 34. More particularly, the sensor 15 can be disposed between the body facing surface 27 of the film layer 26b of the outer cover 26 and the fluid transfer layer 46. Such a configuration may be beneficial in that the sensor 15, and its output mechanism(s), may be viewable by a user from the outer cover 26, even while the subject is wearing the absorbent article 10. Although not in direct contact with the subject's skin, the sensor 15 can be exposed to the microbial community of the sample area through the microbial community being transferred by exudates.

As depicted in FIG. 5B, an alternative embodiment can have the sensor 15 disposed between a garment facing surface 57 of the bodyside liner 28 and the absorbent body 34. More particularly, the sensor 15 can be disposed between a garment facing surface 57 of the bodyside liner 28 and the fluid transfer layer 46. Such a configuration may allow for more discrete viewing in that the sensor 15 can be viewed by a user from the interior of the absorbent article 10 after it is changed, but not from the exterior of the absorbent article 10. As an example, the sensor 15 could be viewed by a user from the body facing surface 55 of the bodyside liner 28 after that absorbent article 10 is changed, but not from a garment facing surface 33 of the outer cover 26 while the absorbent article 10 is being worn. In some embodiments, the sensor 15 can extend from the crotch region 16 to the front waist region 12 (such as shown in FIG. 4) to avoid the potential of the sensor 15 being completely covered with body exudates in the crotch region 16. In some embodiments, the sensor 15 can be configured to extend from the crotch region 16 to the rear waist region 14. In some embodiments, the sensor 15 can be configured to be only located within one respective region of the rear waist region 14, the crotch region 16, or the front waist region 12.

Of course, it is contemplated that the sensor 15 can be included in an absorbent article 10 in alternative configurations than those exemplary embodiments depicted and described above. As but one example, it is contemplated that a sensor 15 of the present disclosure could be disposed on the body facing surface 55 of the bodyside liner 28. Such a configuration may provide the enhanced benefit of having direct contact with the skin of the sample area of the subject and help the detection mechanism(s) and output mechanism(s) in identifying a potential forthcoming skin or gastrointestinal issue for a subject. Additionally, it is to be noted that in some embodiments, the sensor 15 need not be viewable by a user from the outer cover 26 or the bodyside liner 28 to indicate to a user of a potential forthcoming skin or gastrointestinal issue.

Wipe

In some embodiments, the sensor 15 of the present disclosure can be incorporated into or on a wipe. For example, the sensor 15 may be incorporated into or onto a substrate, such as a wipe substrate. In one embodiment, the sensor 15 may be used in combination with a wipe substrate and a wetting composition to form a wet wipe which may be dispersible. In other embodiments, the sensor 15 may be incorporated into wipes such as wet wipes, hand wipes, face wipes, cosmetic wipes, cloths and the like. In yet other embodiments, the sensor 15 described herein can be used in combination with numerous personal care products, such as absorbent articles as described above. As mentioned above, absorbent articles of interest to include a sensor 15 can include, but are not limited to, diapers, training pants, adult incontinence products, feminine hygiene products, and the like. Other personal care products that could be configured to include a sensor 15 as described herein can include, but are not limited to, bath or facial tissue and paper towels. Other personal protective equipment articles of interest to include a sensor 15 can include, but are not limited to, masks, gowns, gloves, caps, and the like.

In one embodiment, a wet wipe may comprise a nonwoven material that is wetted with an aqueous solution termed the “wetting composition.” As used herein, the nonwoven material comprises a fibrous material or substrate, where the fibrous material or substrate comprises a sheet that has a structure of individual fibers or filaments randomly arranged in a mat-like fashion. Nonwoven materials may be made from a variety of processes including, but not limited to, airlaid processes, wet-laid processes such as with cellulosic-based tissues or towels, hydroentangling processes, staple fiber carding and bonding, melt blown, and solution spinning.

When the wetting composition is added to a delivery vehicle, such as a wipe substrate, the amount of add-on of the wetting composition can range from about 100% to about 400%, or more preferably from about 200% to about 375%, or even more preferably from about 240% to about 350%. In some embodiments, the add-on of the wetting composition can be from 200% to about 350%. In one particular embodiment, the add-on can be about 330%.

The fibers forming the fibrous material may be made from a variety of materials including natural fibers, synthetic fibers, and combinations thereof. The choice of fibers may depend upon, for example, the intended end use of the finished substrate and the fiber cost. For instance, suitable fibers may include, but are not limited to, natural fibers such as cotton, linen, jute, hemp, wool, wood pulp, etc. Similarly, suitable fibers may also include: regenerated cellulosic fibers, such as viscose rayon and cuprammonium rayon; modified cellulosic fibers, such as cellulose acetate; or synthetic fibers, such as those derived from polypropylenes, polyethylenes, polyolefins, polyesters, polyamides, polyacrylics, etc. Regenerated cellulose fibers, as briefly discussed above, include rayon in all its varieties as well as other fibers derived from viscose or chemically modified cellulose, including regenerated cellulose and solvent-spun cellulose, such as Lyocell. Among wood pulp fibers, any known papermaking fibers may be used, including softwood and hardwood fibers. Fibers, for example, may be chemically pulped or mechanically pulped, bleached or unbleached, virgin or recycled, high yield or low yield, and the like. Chemically treated natural cellulosic fibers may be used, such as mercerized pulps, chemically stiffened or crosslinked fibers, or sulfonated fibers.

In addition, cellulose produced by microbes and other cellulosic derivatives may be used. As used herein, the term “cellulosic” is meant to include any material having cellulose as a major constituent, and, specifically, comprising at least 50 percent by weight cellulose or a cellulose derivative. Thus, the term includes cotton, typical wood pulps, non-woody cellulosic fibers, cellulose acetate, cellulose triacetate, rayon, thermomechanical wood pulp, chemical wood pulp, debonded chemical wood pulp, milkweed, or bacterial cellulose. lends of one or more of any of the previously described fibers may also be used, if so desired.

The fibrous material may be formed from a single layer or multiple layers. In the case of multiple layers, the layers are generally positioned in a juxtaposed or surface-to-surface relationship and all or a portion of the layers may be bound to adjacent layers. The fibrous material may also be formed from a plurality of separate fibrous materials wherein each of the separate fibrous materials may be formed from a different type of fiber.

Airlaid nonwoven fabrics are particularly well suited for use as wet wipes. The basis weights for airlaid nonwoven fabrics may range from about 20 to about 200 grams per square meter (gsm) with staple fibers having a denier of about 0.5 to about 10 and a length of about 6 to about 15 millimeters. Wet wipes may generally have a fiber density of about 0.025 g/cc to about 0.2 g/cc. Wet wipes may generally have a basis weight of about 20 gsm to about 150 gsm. More desirably the basis weight may be from about 30 to about 90 gsm. Even more desirably the basis weight may be from about 50 gsm to about 75 gsm.

Processes for producing airlaid non-woven basesheets are described in, for example, published U.S. Pat. App. No. 2006/008621, herein incorporated by reference to the extent it is consistent herewith.

### EMBODIMENTS

Embodiment 1: A sensor comprising: a first detection mechanism configured for detecting at least one Bacteroides virulence factor present from a sample location; and an output mechanism configured to provide an indication of a potential forthcoming skin or gastrointestinal issue based on at least one 20 Bacteroides virulence factor being detected.

Embodiment 2: The sensor of embodiment 1, wherein the output mechanism is configured to provide the indication of a potential forthcoming skin or gastrointestinal issue when the first detection mechanism detects at least one Bacteroides virulence factor at an amount of at least 100 pg/mL.

Embodiment 3: The sensor of embodiment 1 or 2, wherein the first detection mechanism is configured for detecting a plurality of Bacteroides virulence factors.

Embodiment 4: The sensor of any one of the preceding embodiments, wherein the at least one Bacteroides virulence factor comprises at least one of: Bacteroides structural targets, human produced targets associated with Bacteroides, Bacteroides produced targets, and Bacteroides genomic elements targets.

Embodiment 5: The sensor of embodiment 4, wherein the Baderoides structural targets comprise capsular polysaccharides, zwitterionic polysaccharides, lipopolysaccharides, pili fimbriae, and Omp 200.

Embodiment 6: The sensor of embodiment 4 or 5, wherein the human produced targets associated with Bacteroides comprise Regllly and antibiotic Paneth cell protein (Ang4).

Embodiment 7: The sensor of any one of embodiments 4-6, wherein the Bacteroides produced targets comprise Hyalunidase and chonodroitin sulfatase, hemolysins (HlyA and HlyB), Neuraminidase, Enterotoxin (BFT), Endotoxin (LPS), and Bacteriocin protein.

Embodiment 8: The sensor of any one of embodiments 4-7, wherein the Bacteroides genomic elements targets comprise a sequence comprising sequence NO. 1, sequence NO. 2, sequence NO. 3, and sequence NO. 4 in the Sequence Listing.

Embodiment 9: The sensor of any one of the preceding embodiments, further comprising: a second detection mechanism configured for detecting an amount of Bacteroides and an amount of Anaerococcus from the sample location; the output mechanism being further configured to provide the indication of a potential forthcoming skin or gastrointestinal issue based on a Bacteroides to Anaerococcus ratio of at least 0.4 being detected.

Embodiment 10: The sensor of any one of the preceding embodiments, wherein the sensor is incorporated into an absorbent article.

Embodiment 11: The absorbent article of embodiment 10, wherein the absorbent article comprises a bodyside liner and an outer cover, and wherein the sensor is disposed between a body-facing surface of the bodyside liner and a garment facing surface of the outer cover.

Embodiment 12: The absorbent article of embodiment 11, wherein the sensor is viewable from the garment facing surface of the outer cover.

Embodiment 13: The sensor of any one of embodiments 1-9, wherein the sensor is incorporated into a wipe.

Embodiment 14: A sensor comprising: a detection mechanism configured for detecting an amount of Bacteroides and an amount of Anaerococcus from the sample location; and an output mechanism configured to provide an indication of a potential forthcoming skin or gastrointestinal issue.

Embodiment 15: The sensor of embodiment 14, wherein the output mechanism is configured to provide an indication of a potential forthcoming skin or gastrointestinal issue based on a Bacteroides to Anaerococcus ratio of at least 0.4 being detected.

Embodiment 16: The sensor of embodiment 14, wherein the output mechanism is configured to provide an indication of a potential forthcoming skin or gastrointestinal issue based on a Bacteroides to Anaerococcus ratio of at least 1.0 being detected.

Embodiment 17: A method for indicating a potential forthcoming skin or gastrointestinal issue in a subject, the method comprising: providing a first sensor, the first sensor comprising: a first detection mechanism configured for detecting at least one Bacteroides virulence factor present from a sample location on the subject; and applying the first sensor to the subject; and indicating the potential forthcoming skin or gastrointestinal issue when at least one Bacteroides virulence factor is detected at a primary sampling time.

Embodiment 18: The method of embodiment 17, wherein indicating the potential forthcoming skin or gastrointestinal issue occurs when the amount of the at least one Bacteroides virulence factor detected by the first detection mechanism is at least 100 pg/mL.

Embodiment 19: The method of embodiment 17 or 18, wherein the first detection mechanism is configured for detecting a plurality of Bacteroides virulence factors.

Embodiment 20: The method of any one of embodiments 17-19, wherein the at least one Bacteroides virulence factor comprises at least one of: Bacteroides structural targets, human produced targets associated with Bacteroides, Bacteroides produced targets, and Bacteroides genomic elements targets.

Embodiment 21: The method of any one of embodiments 17-20, wherein the method further comprises: providing a second sensor, the second sensor comprising: a second detection mechanism configured for detecting Bacteroides and Anaerococcus from the sample location on the subject; applying the second sensor to the subject; wherein the indicating the potential forthcoming skin or gastrointestinal issue is completed when the first detection mechanism detects at least one Bacteroides virulence factor and when the second detection mechanism detects a Bacteroides to Anaerococcus ratio is at least 0.4 at the primary sampling time.

Embodiment 22: The method of any one of embodiments 17-20, the method further comprising: removing the first sensor from the subject after the primary sampling time; providing a third sensor, the third sensor comprising: a third detection mechanism configured for detecting an amount of the at least one Bacteroides virulence factor present from the sample location on the subject; and applying the third sensor to the subject after the first sensor has been removed from the subject after the primary sampling time; and wherein the indicating the potential forthcoming skin or gastrointestinal issue is completed when the amount of the at least one Bacteroides virulence factor detected at a secondary sampling time is greater than the amount of the at least one Bacteroides virulence factor detected by the first detection mechanism at the primary sampling time, the secondary sampling time being after the primary sampling time.

Embodiment 23: The method of claim 22, further comprising: providing a second sensor, the second sensor comprising: a second detection mechanism configured for detecting an amount of Bacteroides and an amount of Anaerococcus to provide a ratio of from the sample location on the subject; applying the second sensor to the subject; removing the second sensor from the subject after the primary sampling time; providing a fourth sensor, the fourth sensor comprising: a fourth detection mechanism configured for detecting an amount of Bacteroides and an amount of Anaerococcus from the sample location on the subject at the secondary sampling time; and applying the fourth sensor to the subject after the second sensor has been removed from the subject after the primary sampling time; and wherein the indicating the potential forthcoming skin or gastrointestinal issue is completed when the amount of the at least one Bacteroides virulence factor detected by the third detection mechanism at the secondary sampling time is greater than the amount of the at least one Bacteroides virulence factor detected by the first detection mechanism at the primary sampling time and when the Bacteroides to Anaerococcus ratio at the secondary sampling time detected by the fourth detection mechanism is greater than the Bacteroides to Anaerococcus ratio at the primary sampling time detected by the second detection mechanism.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A sensor comprising:
a first detection mechanism configured for detecting at least one Bacteroides virulence factor present from a sample location; and
an output mechanism configured to provide an indication of a potential forthcoming skin or gastrointestinal issue based on at least one Bacteroides virulence factor being detected.

2. The sensor of claim 1, wherein the output mechanism is configured to provide the indication of a potential forthcoming skin or gastrointestinal issue when the first detection mechanism detects at least one Bacteroides virulence factor at an amount of at least 100 pg/mL.

3. The sensor of claim 1, wherein the first detection mechanism is configured for detecting a plurality of Bacteroides virulence factors.

4. The sensor of claim 1, wherein the at least one Bacteroides virulence factor comprises at least one of Bacteroides structural targets, human produced targets associated with Bacteroides, Bacteroides produced targets, and Bacteroides genomic elements targets.

5. The sensor of claim 4, wherein the Bacteroides structural targets comprise capsular polysaccharides, zwitterionic polysaccharides, lipopolysaccharides, pili fimbriae, and Omp 200.

6. The sensor of claim 4, wherein the human produced targets associated with Bacteroides comprise Regllly and antibiotic Paneth cell protein (Ang4).

7. The sensor of claim 4, wherein the Bacteroides produced targets comprise Hyalunidase and chonodroitin sulfatase, hemolysins (HlyA and HlyB), Neuraminidase, Enterotoxin (BFT), Endotoxin (LPS), and Bacteriocin protein.

8. The sensor of claim 4, wherein the Bacteroides genomic elements targets comprise a sequence comprising sequence NO. 1, sequence NO. 2, sequence NO. 3, and sequence NO. 4 in the Sequence Listing.

9. The sensor of claim 1, further comprising:
a second detection mechanism configured for detecting an amount of Bacteroides and an amount of Anaerococcus from the sample location;
the output mechanism being further configured to provide the indication of a potential forthcoming skin or gastrointestinal issue based on a Bacteroides to Anaerococcus ratio of at least 0.4 being detected.

10. The sensor of claim 1, wherein the sensor is incorporated into an absorbent article.

11. The absorbent article of claim 10, wherein the absorbent article comprises a bodyside liner and an outer cover, and wherein the sensor is disposed between a body-facing surface of the bodyside liner and a garment facing surface of the outer cover.

12. The absorbent article of claim 11, wherein the sensor is viewable from the garment facing surface of the outer cover.

13. The sensor of claim 1, wherein the sensor is incorporated into a wipe.

14. A sensor comprising:
a detection mechanism configured for detecting an amount of Bacteroides and an amount of Anaerococcus from the sample location; and
an output mechanism configured to provide an indication of a potential forthcoming skin or gastrointestinal issue based on a Baderoides to Anaerococcus ratio.

15. The sensor of claim 14, wherein the output mechanism is configured to provide an indication of a potential forthcoming skin or gastrointestinal issue based on a Bacteroides to Anaerococcus ratio of at least 0.4 being detected.

16. The sensor of claim 14, wherein the output mechanism is configured to provide an indication of a potential forthcoming skin or gastrointestinal issue based on a Bacteroides to Anaerococcus ratio of at least 1.0 being detected.

17. A method for indicating a potential forthcoming skin or gastrointestinal issue in a subject, the method comprising:
providing a first sensor, the first sensor comprising:
a first detection mechanism configured for detecting at least one Bacteroides virulence factor present from a sample location on the subject; and
applying the first sensor to the subject; and
indicating the potential forthcoming skin or gastrointestinal issue when at least one Bacteroides virulence factor is detected at a primary sampling time.

18. The method of claim 17, wherein indicating the potential forthcoming skin or gastrointestinal issue occurs when the amount of the at least one Bacteroides virulence factor detected by the first detection mechanism is at least 100 pg/mL.

19. The method of claim 17, wherein the first detection mechanism is configured for detecting a plurality of Bacteroides virulence factors.

20. The method of claim 17, wherein the at least one Bacteroides virulence factor comprises at least one of Bacteroides structural targets, human produced targets associated with Bacteroides, Bacteroides produced targets, and Bacteroides genomic elements targets.

21. The method of claim 17, wherein the method further comprises:
providing a second sensor, the second sensor comprising:
a second detection mechanism configured for detecting Baderoides and Anaerococcus from the sample location on the subject;
applying the second sensor to the subject; and
wherein the indicating the potential forthcoming skin or gastrointestinal issue is completed when the first detection mechanism detects at least one Bacteroides virulence factor and when the second detection mechanism detects a Bacteroides to Anaerococcus ratio is at least 0.4 at the primary sampling time.

22. The method of claim 17, the method further comprising:
removing the first sensor from the subject after the primary sampling time;
providing a third sensor, the third sensor comprising:
a third detection mechanism configured for detecting an amount of the at least one Bacteroides virulence factor present from the sample location on the subject; and
applying the third sensor to the subject after the first sensor has been removed from the subject after the primary sampling time; and
wherein the indicating the potential forthcoming skin or gastrointestinal issue is completed when the amount of the at least one Bacteroides virulence factor detected at a secondary sampling time is greater than the amount of the at least one Bacteroides virulence factor detected by the first detection mechanism at the primary sampling time, the secondary sampling time being after the primary sampling time.

23. The method of claim 22, further comprising:
providing a second sensor, the second sensor comprising:
a second detection mechanism configured for detecting an amount of Bacteroides and an amount of Anaerococcus to provide a ratio of from the sample location on the subject;
applying the second sensor to the subject;
removing the second sensor from the subject after the primary sampling time;
providing a fourth sensor, the fourth sensor comprising:
a fourth detection mechanism configured for detecting an amount of Bacteroides and an amount of Anaerococcus from the sample location on the subject at the secondary sampling time; and
applying the fourth sensor to the subject after the second sensor has been removed from the subject after the primary sampling time; and
wherein the indicating the potential forthcoming skin or gastrointestinal issue is completed when the amount of the at least one Bacteroides virulence factor detected by the third detection mechanism at the secondary sampling time is greater than the amount of the at least one Bacteroides virulence factor detected by the first detection mechanism at the primary sampling time and when the Bacteroides to Anaerococcus ratio at the secondary sampling time detected by the fourth detection mechanism is greater than the Bacteroides to Anaerococcus ratio at the primary sampling time detected by the second detection mechanism.
